(19) United States
(12) Patent Application Publication
(10) Pub. No.: US 2015/0374713 A1
(43) Pub. Date: Dec. 31, 2015

(54) STABLE PHARMECEUTICAL COMPOSITION OF AMLODIPINE AND BENAZEPRIL OR SALTS THEREOF

(71) Applicant: WOCKHARDT LIMITED, Aurangabad (IN)

(72) Inventors: Girish Kumar JAIN, Delhi 4 (IN); Venkataramana NAIDU, Secunderabad 9 (IN); Pankaj Umakant ATTARDE, Nasik (IN)

(73) Assignee: WOCKHARDT LIMITED, Aurangabad (IN)

(21) Appl. No.: 14/766,216

(22) PCT Filed: Feb. 5, 2014
(86) PCT No.: PCT/IB2014/058796

§ 371 Date: Aug. 6, 2015

(30) Foreign Application Priority Data
Feb. 8, 2013 (IN) 384/MUM/2013

(51) Int. Cl. A61K 31/55; A61K 9/20; A61K 9/28; A61K 9/48; A61K 31/4422

(52) U.S. Cl. CPC A61K 31/55; A61K 9/4808; A61K 31/4422; A61K 9/2833; A61K 9/4833; A61K 9/2095

(57) ABSTRACT

The present invention relates to a stable pharmaceutical composition of amiodipine and benazepril or salts thereof. In particular, the invention relates to a stable pharmaceutical composition comprising at least one amiodipine component and at least two benazepril components in which physical contact between amiodipine and benazepril components is limited. The invention also includes a process of preparing such compositions and method of treating hypertension by administering the composition to a patient in need thereof.

### FIELD OF THE INVENTION

The present invention relates to a stable pharmaceutical composition of amlodipine and benazepril or salts thereof. In particular, the invention relates to a stable pharmaceutical composition comprising at least one amlodipine component and at least two benazepril components in which physical contact between amlodipine and benazepril components is limited. The invention also includes a process of preparing such compositions and method of treating hypertension by administering the composition to a patient in need thereof.

### BACKGROUND OF THE INVENTION

Cardiovascular diseases are a group of disorders of the heart and blood vessels and include coronary heart disease, cerebrovascular disease, peripheral arterial disease, rheumatic heart disease, congenital heart disease, deep vein thrombosis and pulmonary embolism. Heart attacks and strokes are usually acute events and are mainly caused by a blockage that prevents blood from flowing to the heart or brain.

A variety of therapeutic options are currently employed in the treatment of cardiovascular diseases and conditions associated with cardiovascular diseases.

Combination therapy of an angiotensin converting enzyme inhibitors with a calcium channel blocker can achieve synergistic results effective in treating a variety of conditions including hypertension, congestive heart failure, angina, myocardial infarction, atherosclerosis, diabetic nephropathy, and diabetic cardiac myopathy, renal insufficiency, peripheral vascular disease, left ventricular hypertrophy, cognitive dysfunction, stroke, and headache.

Numerous studies have been conducted to address the combination formulation variables including combination therapy of ACE inhibitors and calcium channel blockers, and attempts have been made to improve the combination therapy.

Benazepril is a non-sulfhydryl angiotensin-converting enzyme (ACE) inhibitor. Chemically, Benazepril is 2-[(3S)-3-{[(2S)-1-ethoxy-1-oxo-4-phenylbutan-2-yl]amino}-2-oxo-2,3,4,5-tetrahydro-1H-1-benzazepin-1-yl]acetic acid, with the following structure:

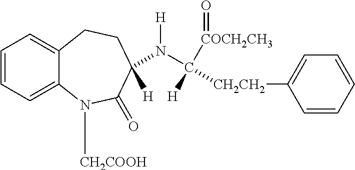

Amlodipine is a long-acting 1,4-dihydropyridine calcium channel blocker, chemically, Amlodipine, is 3-ethyl 5-methyl 2-[(2-aminoethoxy)methyl]-4-(2-chlorophenyl)-6-methyl-1,4-dihydropyridine-3,5-dicarboxylate, with the following structure:

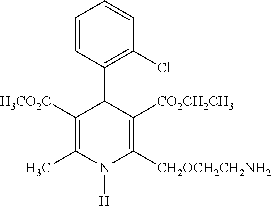

A combination of benazepril hydrochloride and amlodipine besylate is the drug of choice for the treatment of cardiovascular diseases. A combination of benazepril hydrochloride and amlodipine besylate is marketed by Novartis in the United States in the form of oral capsules under trade name Lotrel®.

U.S. Pat. Nos. 4,410,520 and 4,572,909 respectively discloses a benazepril, benazeprilat and amlodipine, their salts along with pharmaceutical dosage forms, dosage ranges and suitable routes of administration therewith, and uses.

U.S. Pat. No. 4,879,303 discloses a besylate salt of amlodipine.

European Patent No. EP 2,574,85 B1 discloses a combination of ACE-inhibiting compounds and inotropic dihydropyridines and is used for lowering blood pressure.

European Patent Application No. EP 3,342,64 A2 discloses composition of an ACE inhibitor alone or in combination with a calcium channel blocker.

U.S. Pat. No. 6,162,802 discloses a combination composition of benazepril and amlodipine. The benazepril and the amlodipine are administered in a single dosage form, such that the benazepril and amlodipine are completely physically separated from each other.

U.S. Pat. No. 8,158,146 discloses a combination formulation of benazepril and amlodipine in which both benazepril and amlodipine are in complete physical contact.

Benazepril and amlodipine are physically incompatible substances. Hence, if incorporated into a single dosage form they must be kept physically separated. This may be accomplished in any of the myriad ways known in the art, such as bi-layered tablets, coated pellets of one agent incorporated into a tablet of the other, separately coated pellets of each agent in a capsule or tablet, coated pellets of one agent in capsule together with powder of the other agent, each agent microencapsulated separately and then blended together for use in a tablet or capsule, use of a dual or multiple compartment transdermal device, etc. Due to the incompatibility, combination products of the two agents in an injectable solution are not really acceptable. For convenience purposes, a coated compressed tablet of benazepril together with amlodipine powder in a capsule has been found to be the most desirable oral form.

It is evident from the prior art that stable formulations of benazepril and amlodipine can be prepared by maintaining complete physical separation due to possible incompatibility between both the actives. The prior also suggests a method of preparing stable combination formulation of both the active in which the actives are in complete physical contact. However, such formulation may not substantiate the ultimate stability of the resulting formulation due to incompatibility between the two actives.

Thus, there still exists a need to devise a novel and alternative formulation of amlodipine and benazepril which could provide a viable alternative to the existing formulations, without compromising on the stability over the storage period and involve simple manufacturing process which is extendable for commercial manufacturing.

The inventors of the present invention have devised a novel pharmaceutical composition comprising at least one amlodipine component and at least two benazepril components such that physical contact between amlodipine and benazepril components is limited. Particularly, the inventors have found that when physical contact between benazepril component and amlodipine component is limited enough, the resulting composition can retain the desired stability over the storage period.

The present invention relates to a novel and stabilized pharmaceutical composition of benazepril and amlodipine and process of preparing such compositions.

### SUMMARY OF THE INVENTION

In one general aspect, there is provided a stable pharmaceutical composition comprising:

(a) at least one first benazepril component comprising benazepril or salts thereof;
(b) at least one second benazepril component comprising benazepril or salts thereof; and
(c) at least one amlodipine component comprising amlodipine or salts thereof,
wherein the first benazepril component is substantially physically separated from the amlodipine component and the second benazepril component is in physical contact with the amlodipine component.

In another general aspect, the second benazepril component is in intimate physical contact with the amlodipine components.

In another general aspect, the second benazepril component and amlodipine components are physically inseparable.

In another general aspect, the second benazepril component and amlodipine components are in the form of a blend, which comprises amlodipine, benazepril or salts thereof and one or more pharmaceutical excipients.

In another general aspect, the second benazepril component is in limited or enough physical contact with at least one amlodipine component such that the composition exhibits desired stability over the storage period.

In another general aspect, first benazepril component is devoid of amlodipine.

In another general aspect, 50% of the total amount of benazepril in the composition is present in the first components and remaining 50% of the benazepril in the composition is present in the second components.

In another general aspect, the stable pharmaceutical composition of the present invention retains at least 90% w/w of total potency of both amlodipine and benazepril after storage at 40° C. and 75% relative humidity for at least 3 months.

In another general aspect, the stable pharmaceutical composition of the present invention comprises no more than 2.5% of (s,s)-diacid by weight relative to the benazepril; and/or no more than 0.3% of impurity D by weight relative to the amlodipine.

In another general aspect, there is provided a capsule comprising:

(a) one or more tablets comprising benazepril or its salt with one or more pharmaceutical excipients; and
(b) a blend comprising benazepril, amlodipine or salts thereof and one or more pharmaceutical excipients.

In another general aspect, there is provided a process for preparation of a stable pharmaceutical composition comprising one or more amlodipine components and at least two benazepril components, which process comprises of:

(a) providing at least one first benazepril component;
(b) providing at least one second benazepril component;
(c) providing at least one amlodipine component; and
(d) formulating the first and second benazepril components in a unit dosage form,
wherein the first benazepril component is substantially physically separated from the amlodipine component and the second benazepril component is in physical contact with the amlodipine component.

In another general aspect, there is provided a method of treating hypertension in a patient which method comprising administering the pharmaceutical composition as substantially described herein.

### DETAILED DESCRIPTION OF THE INVENTION

The stable pharmaceutical composition comprises at least one first benazepril component; at least one second benazepril component; and at least one amlodipine component, wherein the first benazepril component is substantially physically separated from the amlodipine component and the second benazepril component is in physical contact with the amlodipine component. The combination composition is administered in the form of a single dosage form.

The term “amlodipine” as used throughout the specification refers to not only amlodipine per se, but also its pharmaceutically acceptable salts, solvates, hydrates, enantiomers and polymorphs. The preferred salt is amlodipine besylate.

The term “benazepril” as used throughout the specification refers to not only amlodipine per se, but also its pharmaceutically acceptable salts, solvates, hydrates, enantiomers and polymorphs. The preferred salt is benazepril hydrochloride.

The term “substantially physically separated from the amlodipine component” as used throughout the specification refers to less than 10% w/w, preferably less than 5% of amlodipine or salt thereof is in physical contact with second benazepril component.

The term “physical contact” used throughout the specification refers to a physical contact at an uncoated interface or physical contact throughout a blended mixture of the two active ingredients. Particularly, the term “physical contact” is used to refer an intimate physical contact between the two active ingredients.

The term “component” as used throughout the specification refers to one or more tablets, mini-tablets, powder blend, granules, pellets, beads, solid particles, layers coated on a surface or combination thereof.

In an embodiment, the composition comprises one or more tablets comprising first part of benazepril or salt thereof along with one or more pharmaceutical excipients and a powder blend comprising the second part of benazepril or salt thereof, amlodipine or salt thereof and one or more pharmaceutical excipients. The second part of benazepril and amlodipine in the blend are in physical contact with each other.

The stable pharmaceutical composition of the present invention retains at least 90% w/w of total potency of both amlodipine and benazepril after storage at 40° C. and 75% relative humidity for at least 3 months. The composition comprises no more than 2.5% of (s,s)-diacid (which is the main degradation product of benazepril, also known as benazeprilat) by weight relative to the total amount of benazepril; and/or no more than 0.3% of impurity D (which is the main degradation product of amlodipine) by weight relative to the total amount of amlodipine.

In an embodiment, the second benazepril component is in intimate physical contact with the amlodipine components. In an alternate embodiment, the second benazepril component and amlodipine components are physically inseparable.

In another embodiment, first benazepril component is devoid of amlodipine.

In a further embodiment, 50% of the total amount of benazepril in the composition is present in the first components and remaining 50% of the benazepril in the composition is present in the second components.

In an embodiment, the second benazepril component and amlodipine components are in the form of a blend, which comprises amlodipine, benazepril or salts thereof and one or more pharmaceutical excipients.

In a further embodiment, the composition comprises one or more tablets comprising first part of benazepril or salt thereof, amlodipine or salt thereof along with one or more pharmaceutical excipients and a powder blend comprising the second part of benazepril or salt thereof and one or more pharmaceutical excipients. The first part of benazepril and amlodipine in the blend are in physical contact with each other.

The physical separation between the first component of benazepril and amlodipine may be achieved by placing one or more protective coats over either on the first benazepril component or on the part of the composition comprising second benazepril part and amlodipine.

The stale pharmaceutical composition of the present invention is developed in the form of single unit dosage form, such as capsules, multilayer tablets, bead, beadlets, tablet in tablet, or inlay tablet.

In an embodiment, the first benazepril component is in the form of tablets and the second benazepril component and amlodipine components are present in the form of blend.

In one preferred embodiment, the stable unit dosage form comprises:

(a) a coated tablets comprising first part of benazepril and one or more pharmaceutical excipients, and
(b) a blend comprising second part of benazepril or salt thereof, amlodipine or salt thereof along with one or more pharmaceutical excipients, wherein the coated tablet is substantially physically separated from the blend.

The dosages can be conveniently presented in unit dosage form and prepared by suitable methods well known in the pharmaceutical arts.

In one embodiment, a process for the preparation of a stable pharmaceutical composition of the present invention comprises steps of:

(a) providing at least one first benazepril component;
(b) providing at least one second benazepril component;
(c) providing at least one amlodipine component; and
(d) formulating the first and second benazepril components in a unit dosage form.

The stable composition of the present invention may include one or more conventional excipients to enable formation of a present composition. The pharmaceutical excipients includes, but not limited to one or more diluents, binders, disintegrants, glidants, lubricants, plasticizer, opacifying agent, and colorants.

Examples of suitable diluents include, but not limited to microcrystalline cellulose, microfine cellulose, lactose, starch, pregelatinized starch, calcium carbonate, calcium sulfate, sugar, dibasic calcium phosphate dihydrate, tribasic calcium phosphate, mannitol, powdered cellulose and sorbitol.

Examples of suitable binders include, but not limited to carboxymethylcellulose sodium, ethyl cellulose, gelatin, hydroxyethyl cellulose, hydroxypropyl cellulose, hydroxypropyl methyl cellulose, methylcellulose, povidone, pregelatinized starch, and starch.

Examples of suitable disintegrants include, but not limited to carboxymethylcellulose calcium, croscarmellose sodium, crospovidone, microcrystalline cellulose, polacrilin potassium, pregelatinized starch, sodium starch glycolate, and starch.

Examples of suitable glidants include, but not limited to colloidal silicon dioxide, magnesium trisilicate, powdered cellulose, starch and talc.

Examples of suitable lubricants include, but not limited to magnesium stearate, calcium stearate, glyceryl monostearate, glyceryl palmitostearate, hydrogenated castor oil, hydrogenated vegetable oil, mineral oil, polyethylene glycol, sodium lauryl sulfate, sodium stearyl fumarate, stearic acid, talc, and zinc stearate.

Examples of suitable plasticizers include, but not limited to such as glycerin and sorbitol, and an opacifying agent or colorant.

In an embodiment, the stable combination composition of the present invention one or more pharmaceutical excipients selected from the group consisting of pregelatinized starch, lactose monohydrate, starch, crospovidone, and microcrystalline cellulose, povidone, hydrogenated castor oil, mannitol, sodium starch glycolate, microcrystalline cellulose, colloidal silicon dioxide, crospovidone magnesium stearate.

The present invention further provides a method of treating hypertension in a patient which method comprising administering the pharmaceutical composition as substantially described herein.

The present invention is further illustrated by the following examples which are provided merely to be exemplary of the invention and do not limit the scope of the invention. Certain modifications and equivalents will be apparent to those skilled in the art and are intended to be included within the scope of the present invention.

### Example 1

### Capsule of Amlodipine Besylate and Benazepril Hydrochloride

TABLE 1

| Sr. | | 2.5 + 10 mg |
|---|---|---|
| No. | BENAZEPRIL HCl tablet | (mg/Tablet) |
| 1 | Benazepril hydrochloride | 5.00 |
| 2 | Lactose Monohydrate | 36.00 |
| 3 | Microcrystalline cellulose | 19.55 |
| 4 | Pregelatinised starch | 3.50 |
| 5 | Purified water | 0.00 |
| 6 | Crospovidone | 3.50 |
| 7 | Colloidal silicon dioxide | 0.35 |
| 8 | Hydrogenated castor oil | 2.10 |
| 9 | Opadry coating | 2.10 |
| | Weight of tablet | 72.10 |
| | Amlodipine Besylate + | |
| | Benazepril HCl blend | (mg/Capsule) |
| 10 | Amlodipine besylate | 3.47 |
| 11 | Benazepril hydrochloride | 5.00 |
| 12 | Microcrystalline cellulose | 88.00 |
| 13 | Mannitol | 37.23 |
| 14 | Sodium starch glycolate | 4.20 |
| 15 | Colloidal silicon dioxide | 1.05 |
| 16 | Talc | 1.05 |
| | Total of blend part | 140.00 |
| | Total fill weight of capsule | 212.10 |

### Process:

### (a) Process for Coated Tablet Preparation:

Half of the total quantity of benazepril hydrochloride, lactose monohydrate, microcrystalline cellulose and pregelatinised starch were mixed with purified water to form granulate. Granulate was dried in fluidized bed dryer at 60° C. till the desired LOD is achieved. To the dry granulate, crospovidone & colloidal silicon dioxide was added followed by lubrication with magnesium stearate. The lubricated granules were then compressed in to tablets. The tablets were then coated using opadry as a coating material.

### (b) Process for Powder Blend Preparation:

A blend of Amlodipine besylate, remaining half of the total quantity of benazepril hydrochloride, microcrystalline cellulose, mannitol, sodium starch glycollate, colloidal silicon dioxide and talc was mixed in a blender.

### (c) Process for Capsule Filling:

The empty hard gelatin capsules were filled by using capsule filling machine by placing the powder blend [prepared in step (b)] and coated tablet [prepared in step (a)] in size ‘1’ capsule shells.

### Example 2

### Stability Study of Amlodipine Besylate and Benazepril Hydrochloride Capsule According to Example 1

TABLE 2

| | | | 40° ± 2° C./ | 25° ± 2° C./ |
|---|---|---|---|---|
| | | | 75% ± 5 RH | 60% ± 5 RH |
| | | Initial | 3 M | 3 M |
| Details | | | | |
| % | Amlodipine | 99.2 | 95.9 | 98.4 |
| Assay | Benazepril | 97.4 | 96.2 | 99.9 |

| Water content (%) | 3.38 | | 3.82 | | 3.82 | |
|---|---|---|---|---|---|---|
| Related Substances (%) | | | | | | |
| Amlodipine Impurity D | 0.020 | | 0.001 | | 0.001 | |
| Amlodipine Highest | 0.009 | | 0.029 | | 0.021 | |
| Unknown | | | | | | |
| Benazepril Impurity [(s,s)- | 0.019 | | 1.444 | | 0.103 | |
| diacid] | | | | | | |
| Benazepril Highest | 0.033 | | 0.027 | | 0.028 | |
| Unknown | | | | | | |
| Total Impurities | 0.283 | | 0.340 | | 0.290 | |
| (Excluding Benazepril | | | | | | |
| related compound C) | | | | | | |
| Dissolution (0.01N HCl, | AMD | BZP | AMD | BZP | AMD | BZP |
| 500 ml, USP \| (Basket), 100 | | | | | | |
| rpm) | | | | | | |
| Not less than 80% of the | 99 | 99 | 102 | 99 | 97 | 100 |
| labeled amount of | | | | | | |
| Benazepril HCl is dissolved | | | | | | |
| in 30 minutes. | | | | | | |
| Not less than 80% of the | | | | | | |
| labeled amount of | | | | | | |
| Amlodipine free base is | | | | | | |
| dissolved in 30 minutes | | | | | | |

Result of the stability study conducted on the capsule of Example 1 indicated that amlodipine besylate and benazepril hydrochloride composition in accordance with the present invention exhibits excellent storage stability over the storage period.

1. A stable pharmaceutical composition comprising:
(a) at least one first benazepril component comprising benazepril or salts thereof;
(b) at least one second benazepril component comprising benazepril or salts thereof; and
(c) at least one amlodipine component comprising amlodipine or salts thereof,
wherein the first benazepril component is substantially physically separated from the amlodipine component and the second benazepril component is in physical contact with the amlodipine component.

2. The stable pharmaceutical composition of claim 1, wherein the second benazepril component is in intimate physical contact with the amlodipine components.

3. The stable pharmaceutical composition of claim 1 or 2, wherein the second benazepril component and amlodipine components are physically inseparable.

4. The stable pharmaceutical composition of claim 1, wherein the first benazepril component comprises one or more protective coats over the said component.

5. The stable pharmaceutical composition of claim 1, wherein the second benazepril component and amlodipine components are in the form of a blend, which comprises amlodipine, benazepril or salts thereof and one or more pharmaceutical excipients.

6. The stable pharmaceutical composition of claim 5, wherein the blend of second benazepril component and amlodipine component is coated with one or more protective coats.

7. The stable pharmaceutical composition of claim 1, 2 or 3, wherein the second benazepril component and amlodipine components are formulated in the form of a tablet comprising benazepril and amlodipine or salts thereof and one or more pharmaceutical excipients and the first benazepril component is in the form of a blend.

8. The stable pharmaceutical composition of claim 1 or 4, wherein the first benazepril component is devoid of amlodipine.

9. The stable pharmaceutical composition of claim 1, wherein 50% of the total amount of benazepril in the composition is present in the first components and remaining 50% of the benazepril in the composition is present in the second components.

10. The stable pharmaceutical composition of any of the preceding claims, wherein the components are in the form of tablets, mini-tablets, powder blend, granules, pellets, beads, solid particles, layers coated on a surface or combination thereof.

11. The stable pharmaceutical composition of any of the preceding claims, wherein the composition retains at least 90% w/w of total potency of both amlodipine and benazepril after storage at 40° C. and 75% relative humidity for at least 3 months.

12. The stable pharmaceutical composition of any of the preceding claims, wherein the composition comprises no more than 2.5% of (s,s)-diacid by weight relative to the total amount of benazepril; and/or no more than 0.3% of impurity D by weight relative to the total amount of amlodipine.

13. A process for preparation of the stable pharmaceutical composition of any of the preceding claims, which process comprises steps of:
(a) providing at least one first benazepril component;
(b) providing at least one second benazepril component;
(c) providing at least one amlodipine component; and
(d) formulating the first and second benazepril components in a unit dosage form,
wherein the first benazepril component is substantially physically separated from the amlodipine component and the second benazepril component is in physical contact with the amlodipine component.

14. A capsule comprising:
(a) one or more tablets comprising benazepril or salts thereof with one or more pharmaceutical excipients; and
(b) a blend comprising benazepril, amlodipine or salts thereof and one or more pharmaceutical excipients.
